(19) United States
(12) Patent Application Publication
(10) Pub. No.: US 2026/0277010 A1
(43) Pub. Date: Sep. 17, 2026

(54) LIGHT SOURCE APPARATUS

(71) Applicant: SONY GROUP CORPORATION, Tokyo (JP)

(72) Inventors: Keisuke HOMMA, Kanagawa (JP); Takeharu TAKASAWA, Kanagawa (JP)

(73) Assignee: SONY GROUP CORPORATION, Tokyo (JP)

(21) Appl. No.: 18/871,788

(22) PCT Filed: Jun. 5, 2023
(86) PCT No.: PCT/JP2023/020820

§ 371 Date: Dec. 4, 2024

(30) Foreign Application Priority Data
Jun. 15, 2022 (JP) 2022-096759

(51) Int. Cl. G02B 27/09; G02B 6/293; G02B 27/14; G02B 27/28; H01S 5/40

(52) U.S. Cl. CPC G02B 27/0922; G02B 6/2938; G02B 27/141; G02B 27/283; H01S 5/4012

(57) ABSTRACT

A light source apparatus according to an embodiment of the present disclosure includes: a first light source unit that outputs first light having a wavelength in a visible light region; a second light source unit that is disposed before the first light source unit, and outputs second light having a wavelength in a near-infrared region; and a light guide on which the first light and the second light are incident. The first light and the second light are incident on the light guide in a coaxial optical path.

### TECHNICAL FIELD

The present disclosure relates to a light source apparatus including, for example, an IR light source as a light source.

### BACKGROUND ART

For example, Patent Literature 1 discloses an illumination device including a pair of lenses that homogenize an illuminance distribution of light outputted from a plurality of light emitting units, and a diffuser plate that increases an emission angle of light outputted from a pair of lenses, between a light source having a plurality of light emitting units and a light guide.

### CITATION LIST

### Patent Literature

Patent Literature 1: Japanese Unexamined Patent Application Publication No. 2014-153697

### SUMMARY OF THE INVENTION

Incidentally, for example, in a light source apparatus using an IR light source that outputs near-infrared light, a top-hat high-angle distribution and an illuminance distribution are demanded to be achieved.

It is desirable to provide a light source apparatus that makes it possible to achieve a top-hat high-angle distribution and a top-hat illuminance distribution.

A light source apparatus according to one embodiment of the present disclosure includes: a first light source unit that outputs first light having a wavelength in a visible light region; a second light source unit that is disposed before the first light source unit, and outputs second light having a wavelength in a near-infrared region; and a light guide on which the first light and the second light are incident. The first light and the second light are incident on the light guide in a coaxial optical path.

In the light source apparatus according to one embodiment of the present disclosure, the first light source unit and the second light source unit that respectively output the first light having the wavelength in the visible light region and the second light having the wavelength in the near-infrared region to be incident on the light guide are disposed before the first light source unit to cause an optical path length of the second light to be longer than an optical path length of the first light, and to cause the first light and the second light to be incident on the light guide coaxially. This increases a divergence angle of the second light.

### BRIEF DESCRIPTION OF THE DRAWINGS

FIG. 1 is a schematic diagram illustrating a configuration example of a light source apparatus according to an embodiment of the present disclosure.

FIG. 2 is a schematic plan diagram illustrating an example of a configuration of a wavelength conversion element illustrated in FIG. 1.

FIG. 3 is an exploded perspective diagram illustrating an example of a structure of a light source illustrated in FIG. 1.

FIG. 4A is an angular distribution diagram of IR light at an LD light emitting point.

FIG. 4B of an angular distribution of the IR light at a condensing point of a condenser lens disposed before a rod integrator.

FIG. 5 is a schematic diagram illustrating an example of a method of cooling a heat source in the light source apparatus illustrated in FIG. 1.

FIG. 6 is a schematic diagram illustrating a configuration example of a light source apparatus according to a modification example of the present disclosure.

### MODES FOR CARRYING OUT THE INVENTION

Hereinafter, embodiments of the present disclosure will be described in detail with reference to the drawings. The following description is a specific example of the present disclosure, but the present disclosure is not limited to the following embodiment. Moreover, the present disclosure does not limit the disposition, dimensions, dimension ratios, and the like of respective components illustrated in the drawings thereto. It is to be noted that the description is given in the following order.
1. Embodiment (an example in which an IR light source is disposed before a visible light source, and visible light and IR light are incident on a light guide coaxially)
2. Modification Examples (other examples of a configuration of the light source apparatus)

### 1. Embodiment

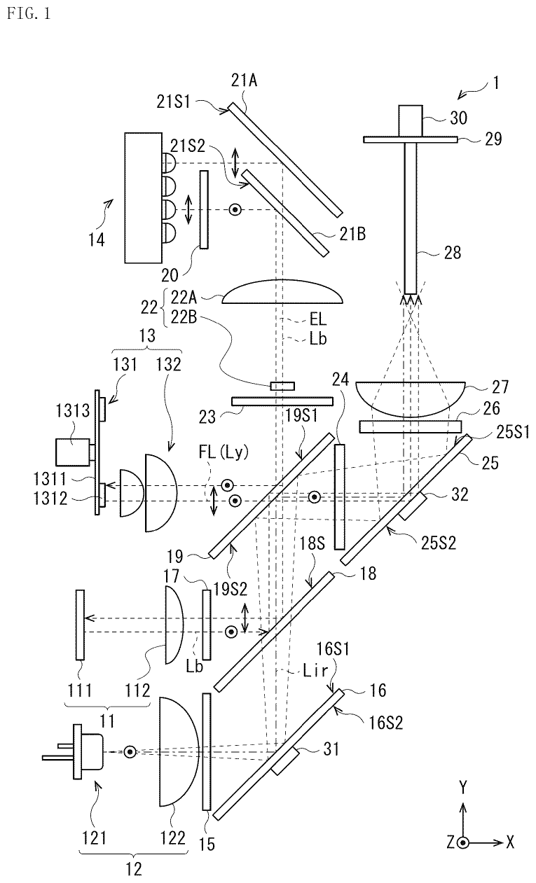

FIG. 1 illustrates a configuration example of a light source apparatus (a light source apparatus 1) according to an embodiment of the present disclosure. The light source apparatus 1 is used, for example, as a light source of a medical device such as a gastric camera or an endoscope camera. The light source apparatus 1 of the present embodiment includes a light source unit 11 that outputs light having a wavelength in a visible light region (for example, a blue light Lb) as a light source, a light source unit 12 that outputs light having a wavelength in a near-infrared region (IR light Lir), and a light guide 30. The light source unit 12 is disposed before the light source unit 11, and is configured such that the blue light Lb and IR light Lir enter the light guide in a coaxial optical path.

### [Configuration of Light Source Apparatus]

The light source apparatus 1 includes, for example, light source units 11 and 12 and a light guide 30. The light source apparatus 1 further includes light source units 13 and 14, beam shaping elements 15 and 23, reflection mirrors 16, 21A, and 25, quarter wavelength plates 17 and 26, a dichroic mirror 18, polarizing beam splitter (PBS) mirrors 19 and 21, a half wavelength plate 20, a reduction lens 22, a polarizing plate 24, a condenser lens 27, a rod integrator 28, and a diffuser plate 29.

Each member constituting the light source apparatus 1 is disposed as follows. The light source units 11 and 12 are disposed in parallel in the order of the light source unit 12 and the light source unit 11 in a Y-axis direction, for example, in a direction (for example, the Y-axis direction) in which pieces of light (the blue light Lb and the IR light Lir) respectively outputted therefrom are respectively reflected by the reflection mirror 16 and the dichroic mirror 18. The beam shaping element 15, the reflection mirrors 16 and 25, the dichroic mirror 18, the PBS mirrors 19 and 21B, the condenser lens 27, the quarter wavelength plate 26, and the rod integrator 28 are disposed between the light source unit 12 and the light guide 30 (on the optical path of the IR light Lir to be outputted from the light source unit 12) in the order of the reflection mirror 16, the dichroic mirror 18, the PBS mirror 19, the polarizing plate 24, the reflection mirror 25, the quarter wavelength plate 26, the condenser lens 27, and the rod integrator 28. The quarter wavelength plate 17 is disposed between the light source unit 11 and the dichroic mirror 18. The light source unit 13 outputs yellow light Ly by wavelength-conversion, and is disposed after the light source unit 11 so as to oppose the PBS mirror 19 in an X-axis direction.

The light source unit 14 outputs the blue light Lb outputted from the light source unit 11 and excitation light EL to be subjected to the wavelength-conversion by the light source unit 13. The light source unit 14 is disposed outside the optical path of the blue light Lb outputted from the light source unit 11 and the IR light Lir outputted from the light source unit 12, such that a part of the outputted light (the blue light Lb) is reflected by the reflection mirror 21A and the dichroic mirror 18 and enters the light source unit 11, and such that a part of the outputted light (the excitation light EL) is reflected by the PBS mirror 21B and 19 and enters the light source unit 13. The reflection mirror 21A, the PBS mirror 21B, the reduction lens 22, and the beam shaping element 23 are disposed in this order between the light source unit 14 and the PBS mirror 19 (on an optical path of the excitation light EL outputted from the light source unit 14).

The light source unit 11 corresponds to a specific example of a “first light source unit” of the present disclosure. The light source unit 11 includes, for example, a diffusion reflection plate 111 that reflects a part of the light outputted from the light source unit 14 as the blue light Lb, a lens 112 that is disposed to oppose the diffusion reflection plate 111, and the quarter wavelength plate 17. The diffusion reflection plate 111 diffuses the blue light Lb reflected by the dichroic mirror 18 and incident through the quarter wavelength plate 17 and the lens 112, and reflects the diffused light toward the lens 112. The lens 112 is, for example, a collimating lens, and adjusts the blue light Lb reflected from the diffusion reflection plate 111 into a collimated light and outputs the collimated light toward the quarter wavelength plate 17. The quarter wavelength plate 17 corrects a polarization state of passing light, and generates a phase difference of quarter wavelength with respect to the passing light. Thus, the blue light Lb polarized in the P-polarized light outputted from the light source unit 14 and reflected by the dichroic mirror 18 toward the diffusion reflection plate 111 is reflected by the diffusion reflection plate 111, is polarized in S-polarized light when passing through the quarter wavelength plate 17, and is reflected by the dichroic mirror 18 toward the PBS mirror 19.

The light source unit 12 corresponds to a specific example of a “second light source unit” of the present disclosure. The light source unit 12 includes a light emitting element 121 and a lens 122 disposed to oppose to the light emitting element 121. As described above, the light emitting element 121 is a solid-state light source that outputs, for example, light having a wavelength in the near-infrared region (Lir). It is possible to use, for example, a semiconductor laser (Laser Diode: LD) that outputs light polarized to S-polarized light or P-polarized light (here, the S-polarized light). Besides, a light-emitting diode (Light Emitting Diode: LED) may be used. The lens 122 is, for example, a collimating lens, and adjusts the IR light Lir reflected from the light emitting element 121 into collimated light and outputs the collimated light toward the reflection mirror 16.

The light source unit 13 corresponds to a specific example of a “third light source unit” of the present disclosure. The light source unit 13 includes a wavelength conversion element 131 and one or more lenses 132. From the light source unit 13, the yellow light Ly adapted to combine the blue light Lb outputted from the light source unit 12 and synthesize white light is outputted.

The wavelength conversion element 131 absorbs (the excitation light EL) outputted from the light source unit 14, converts the light into light having a different wavelength band (for example, the yellow light Ly), and outputs the light. The wavelength conversion element 131 is a so-called reflective wavelength conversion element, and is configured to reflect and output the yellow light Ly generated by the incidence of the excitation light EL.

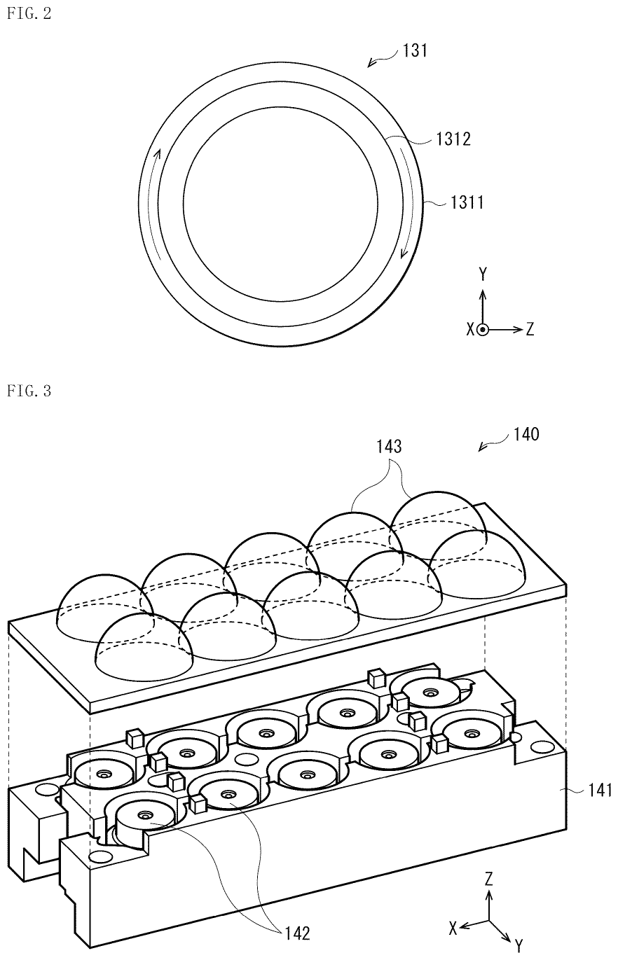

FIG. 2 schematically illustrates an example of a planar configuration of the wavelength conversion element 131. The wavelength conversion element 131 includes, for example, a wheel substrate 1311 that is rotatable about a rotation axis, and a phosphor layer 1312 provided on the wheel substrate 1311.

The wheel substrate 1311 is adapted to support the phosphor layer 1312, and has, for example, a circular plate shape. The wheel substrate 1311 preferably further has a function as a heat dissipation member. Therefore, the wheel substrate 1311 preferably includes a metal material having a high thermal conductivity. Further, it is preferable to use a metal material or a ceramic material that allows for mirror finishing. As a result, it is possible to suppress a temperature increase of the phosphor layer 1312 and improve an extraction efficiency of fluorescence.

Examples of the metal material include an elemental metal such as aluminum (Al), copper (Cu), molybdenum (Mo), tungsten (W), cobalt (Co), chromium (Cr), platinum (Pt), tantalum (Ta), lithium (Li), zirconium (Zr), ruthenium (Ru), rhodium (Rh), and palladium (Pd), or an alloy containing one or more of these metals. Examples of the ceramic material include a material containing silicon carbide (SiC), aluminum nitride (AIN), beryllium oxide (BeO), a composite material of Si and SiC, or a composite material of SiC and Al (with a SiC content of 50% or more).

The phosphor layer 1312 includes a plurality of phosphor particles, and is excited by the excitation light EL and emits the fluorescence FL (e.g., the yellow light Ly) in a wavelength band that differs from the wavelength band of the excitation light EL. The phosphor layer 1312 is formed in, for example, a plate shape, and is configured by, for example, a so-called ceramic phosphor or a binder-type phosphor. The phosphor layer 1312 is continuously formed on the wheel substrate 1311, for example, in a rotational circumferential direction.

Specifically, the phosphor layer 1312 includes phosphor particles that are excited by the excitation light EL outputted from the light source unit 14 and emits the fluorescence FL (the yellow light Ly) in a wavelength band corresponding to yellow. Such phosphor particles include, for example, an YAG (yttrium-aluminum-garnet) based material. The phosphor layer 1312 may further include semiconductor nanoparticles such as quantum dots, organic dyes, or the like.

For example, a motor 1313 is attached to the center of the wheel substrate 1311. The motor 1313 is adapted to rotationally drive the wheel substrate 1311 at a predetermined rotational speed. As a result, the wheel substrate 1311 becomes rotatable, and a position of the excitation light EL applied to the phosphor layer 1312 is temporally changed (moved) at a speed corresponding to the rotational speed. This makes it possible to avoid a deterioration of the phosphor particles caused by a long-time irradiation of the excitation light to the same position of the phosphor layer 1312.

The lens 132 includes one or a plurality of lenses, and includes, for example, a condenser lens and a collimating lens. The lens 132 condenses the excitation light EL reflected by the PBS mirror 19 toward the wavelength conversion element 131, converts the fluorescence FL (the yellow light Ly) outputted from the wavelength conversion element 131 into collimated light, and outputs the collimated light toward the PBS mirror 19.

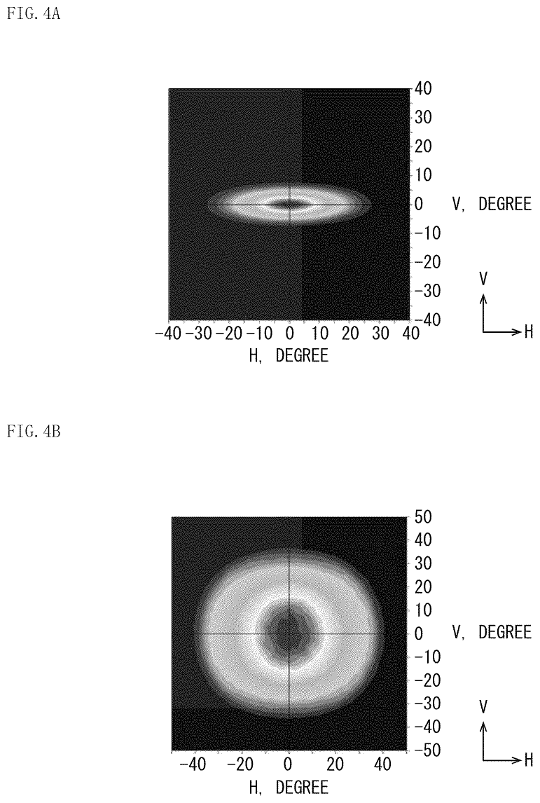

The light source unit 14 includes, as a light source, one or a plurality of solid-state light-emitting elements 142 that output light of a predetermined wavelength band. The one or more solid-state light-emitting elements 142 are disposed, for example, in an array on a base 141. The light source unit 14 corresponds to a specific example of a “fourth light source unit” of the present disclosure. FIG. 3 is an exploded perspective diagram illustrating an example of a configuration of the light source unit 14. In the light source unit 14 illustrated in FIG. 1, for example, ten solid-state light-emitting elements 142 are disposed at the base 141 in, for example, five rows and two columns.

The base 141 supports the plurality of solid-state light-emitting elements 142 and promotes a heat dissipation of the plurality of solid-state light-emitting elements 142 generated by light emission. Therefore, the base 141 preferably includes a material having a high thermal conductivity, and includes, for example, aluminum (Al), copper (Cu), iron (Fe), or the like.

For example, an LD is used for the plurality of solid-state light-emitting elements 142. Specifically, for example, an LD that oscillates laser light (the excitation light EL: the blue light Lb) in a wavelength band corresponding to a blue color of a wavelength from 400 nm to 470 nm is used. The laser light outputted from the plurality of solid-state light-emitting elements 142 is polarized to the S-polarized light or the P-polarized light, and is here polarized to P-polarized light, for example. Besides, LEDs may be used as the plurality of solid-state light-emitting elements 142.

A plurality of lenses 143 is disposed above the plurality of solid-state light-emitting elements 142, respectively. The plurality of lenses 143 is, for example, collimating lenses, and adjusts and outputs the laser light (the excitation light EL) outputted from the plurality of solid-state light-emitting elements 142 into the collimated light. The plurality of lenses 143 is disposed in an array as illustrated in FIG. 3, for example, and is fixed to the base 141 via an adhesive.

The beam shaping element 15 adjusts an angular distribution of the incident laser light and outputs the laser light. Specifically, the laser light has the angular distribution in which a radiation angle difference between a slow axis (an H direction) and a fast axis (a V direction) is large and an LD emission point is largely deviated as illustrated in FIG. 4A. The beam shaping element 15 is, for example, a diffuser plate, and is disposed between the lens 122 and the reflection mirror 16, and outputs the laser beam (IR light Lir) outputted from the light source unit 12 toward the reflection mirror 16 while reducing the angular deviation.

The reflection mirror 16 is disposed after the beam shaping element 15 and reflects the IR light Lir outputted from the light source unit 12 in a predetermined direction. Specifically, the reflection mirror 16 reflects the IR light Lir outputted from the light source unit 12 in the X-axis direction in the Y-axis direction, and is disposed to oppose the light source unit 12 such that a reflection surface 16S1 is at an angle of approximately 45° with respect to an output surface of the light source unit 12.

The dichroic mirror 18 corresponds to a specific example of a “first synthesis mirror” of the present disclosure. The dichroic mirror 18 has a property of selectively reflecting color light of a predetermined wavelength band and transmitting light of another wavelength band. Specifically, the dichroic mirror 18 selectively reflects the blue light Lb. The dichroic mirror 18 is disposed to oppose to the light source unit 11 such that a reflection surface 18S is approximately 45° with respect to an emission surface of the light source unit 11, and is disposed on the optical path of IR light Lir reflected by the reflection mirror 16. As a result, the IR light Lir and the blue light Lb are multiplexed in the coaxial optical path.

The PBS mirror 19 corresponds to a specific example of a “second synthesis mirror” of the present disclosure. The PBS mirror 19 has a polarized selection surface (a surface 19S1), and selects (reflects) light having a predetermined polarization component (for example, an S-polarization component) on the polarization selection surface. Similarly to the dichroic mirror 18, the PBS mirror 19 has a property of selectively reflecting color light in a predetermined wavelength band and transmitting light of another wavelength band. The PBS mirror 19 is disposed to oppose to the light source unit 13 so that a polarization selection surface (a surface 19S1) is at an angle of approximately 45° with respect to an emission surface of the light source unit 13, and is disposed on the optical paths of the IR light Lir and the blue light Lb multiplexed in the dichroic mirror 18.

As a result, a portion of the light outputted from the light source unit 14 (for example, the excitation light EL of the S-polarized light) is reflected toward the light source unit 13, and the remaining light (for example, the blue light Lb of the P-polarized light) is transmitted through the PBS mirror 19 and is reflected toward the light source unit 11 in the dichroic mirror 18. The IR light Lir and the blue light Lb multiplexed in the dichroic mirror 18 are respectively reflected in the X-axis direction on the surfaces 19S1 and 19S2 of the PBS mirror 19. Further, the yellow light Ly outputted from the light source unit 13 transmits through the PBS mirror 19 and is multiplexed in an optical path coaxial with the IR light Lir and the blue light Lb reflected in the X-axis direction.

The half wavelength plate 20 generates a phase difference of approximately ½ wavelength with respect to pieces of light of polarization components orthogonal to each other, and is disposed so as to oppose a portion of an emission surface of the light source unit 14 so as to convert a portion of the light polarized into, for example, P-polarized light outputted from the light source unit 14 into the S-polarized light and output the S-polarized light as the excitation light EL.

The reflection mirror 21A reflects the light (the blue light Lb) outputted from the light source unit 14 in a predetermined direction. Specifically, the reflection mirror 21A reflects the blue light Lb outputted from the light source unit 14 in the X-axis direction in the Y-axis direction, and is disposed to oppose the light source unit 14 such that the reflection surface 21S1 is at an angle of approximately 45° with respect to the emission surface of the light source unit 14.

The PBS mirror 21B reflects the excitation light EL outputted from the light source unit 14 and polarized into the S-polarized light by the half wavelength plate 20 in a predetermined direction. Specifically, the PBS mirror 21B reflects the excitation light EL outputted from the light source unit 14 in the X-axis direction in the Y-axis direction, and is disposed to oppose the light source unit 14 such that the reflection surface 21S2 is at an angle of approximately 45° with respect to the emission surface of the light source unit 14. Further, the blue light Lb outputted from the light source unit 14 and reflected in the Y-axis direction by the reflection mirror 21A as the P-polarized light as it is transmits through the PBS mirror 21B, and is outputted toward the reduction lens 22 together with the excitation light EL.

The reduction lens 22 is disposed between PBS mirror 21B and the beam shaping element 23, reduces the blue light Lb and the excitation light EL outputted from the light source unit 14 and reflected by the reflection mirror 21A and the PBS mirror 21B, and outputs the light toward the beam shaping element 23. The reduction lens 22 has a configuration in which a plurality of lenses (here, lens 22A and 22B) is combined.

The beam shaping element 23 is, for example, a diffuser plate, and adjusts the blue light L and the excitation light EL outputted from the reduction lens 22 to a top-hat illumination distribution and outputs the pieces of light toward the PBS 19.

The polarizing plate 24 corresponds to a specific example of a “polarizing optical element” of the present disclosure. The polarizing plate 24 includes a polarizer having a polarization axis in a predetermined direction, and adjusts the polarization of the IR light Lir disturbed by passing through the beam shaping element 15, the reflection mirror 16, the dichroic mirror 18, and the PBS mirror 19.

The reflection mirror 25 is disposed between the polarizing plate 24 and the quarter wavelength plate 26, and reflects the multiplexed IR light Lir, blue light Lb, and yellow light Ly in a predetermined direction. Specifically, the reflection mirror 25 reflects the IR light Lir, the blue light Lb, and the yellow light Ly outputted in the X-axis direction by the PBS mirror 19, and is disposed such that the reflection surface 21S1 is substantially parallel to the reflection surface 18S1 and 18S2 of the PBS mirror 19.

The quarter wavelength plate 26 corresponds to a specific example of a “polarizing optical element” of the present disclosure. The quarter wavelength plate 26 corrects polarization states of the IR light Lir, the blue light Lb, and the yellow light Ly reflected by the reflection mirror 25, and generates a phase difference of approximately ¼ wavelength with respect to the pieces of light of the polarization components orthogonal to each other. This reduces the deviation of the polarization of the IR light Lir, the blue light Lb, and the yellow light Ly.

The condenser lens 27 corresponds to a specific example of “one or more condenser lenses” of the present disclosure. The condenser lens 27 is disposed before the light guide 30, specifically, between the quarter wavelength plate 26 and the rod integrator 28, and condenses the IR light Lir, the blue light Lb, and the yellow light Ly in which the deviation of the polarization is reduced by the quarter wavelength plate 26 toward the incidence surface of the rod integrator. The condenser lens 27 preferably includes, for example, a high refractive index material. Alternatively, the condenser lens 27 is preferably configured by a combination of a plurality of lenses. As a result, a converging point is reduced, and light amounts of the IR light Lir, the blue light Lb, and the yellow light Ly entering the rod integrator 28 are increased.

The rod integrator 28 corresponds to a specific example of an “integrator optical system” of the present disclosure. The rod integrator 28 uniformizes a non-uniform energy distribution of the incident light. Specifically, the rod integrator 28 enhances a uniformity of the non-uniform illuminance distributions of the IR light Lir, the blue light Lb, and the yellow light Ly condensed by the condenser lens 27. Thereby, the top-hat illuminance distribution of the light applied from the light guide 30 is achieved.

The diffuser plate 29 increases emission angles of the IR light Lir, the blue light Lb, and the yellow light Ly outputted from the rod integrator 28 and causes the pieces of light to enter the light guide 30.

The light guide 30 is adapted to efficiently propagate the light incident from the light source apparatus 1 to the other. It is possible to use, for example, an optical fiber, for the light guide 30.

In the light source apparatus 1, for example, sensors 31 and 32 that detect an amount of light and chromaticity may be disposed on the surface 16S2 and 25S2 of the respective reflection mirrors 16 and 25 which are on an opposite side of the reflection surfaces 16S1 and 25S1. As a result, a detection accuracy of the light amount and the chromaticity is improved as compared with a case where a sensor is disposed outside the light source apparatus 1 to detect the reflected light. Further, it is possible to reduce a size of a sensing mechanism.

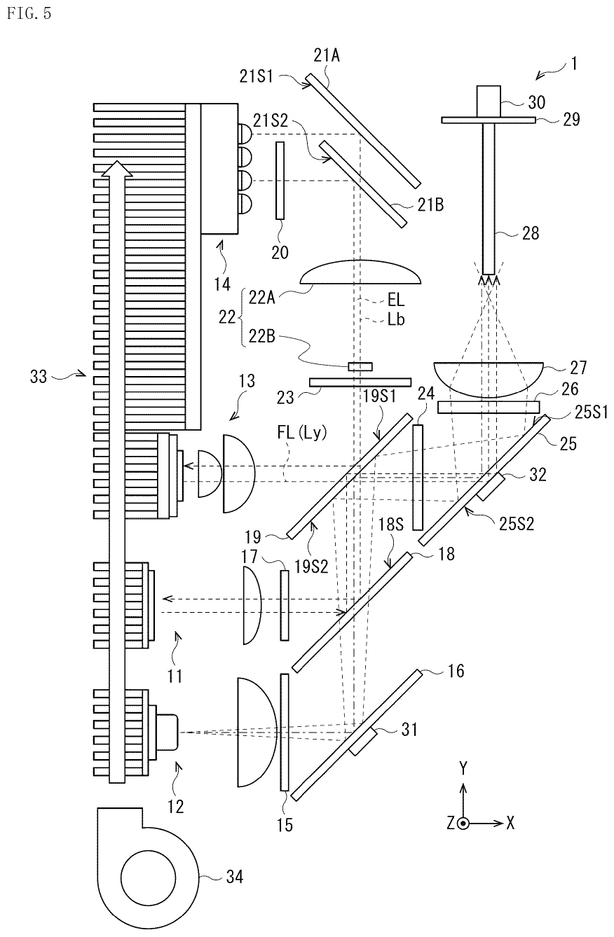

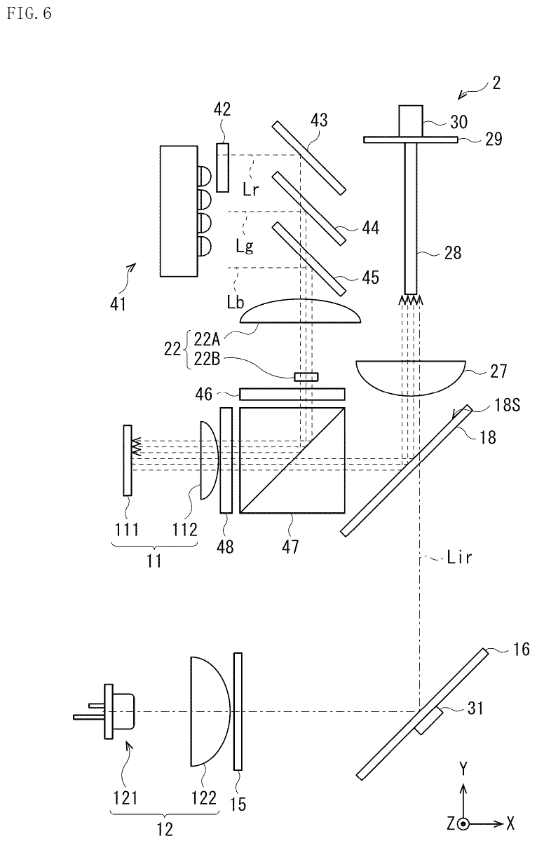

Further, the light source units 11, 12, 13, and 14 serving as heat sources inside the light source apparatus 1 are preferably disposed linearly as illustrated in FIG. 1. For example, as illustrated in FIG. 5, a heat sink 33 may be disposed on a back surface of each light source unit 11. As a result, the cooling efficiency is improved because a path of cooling air blown from the fan 34 becomes linear. Further, as compared with a case where the light source units 11, 12, 13, and 14 are randomly disposed, it is possible to reduce the number of installation fans. Further, in the present embodiment, the light source unit 12 as an IR light source is disposed before the light source unit 11 as a blue light source, making it possible to dispose the light source unit 12, which is high in temperature sensitivity of output, above the air of the cooling air blown from the fan 34.

### [Workings and Effects]

In the light source apparatus 1 of the present embodiment, the light source unit 12 that outputs the light having a wavelength in the near-infrared region (the IR light Lir) is disposed before the light source unit 11 that outputs the light having a wavelength in the visible-light region (for example, the blue light Lb), and the IR light Lir and the blue light Lb are incident on the light guide 30 in a coaxial optical path. This will be described below.

In recent years, because Indocyanine Green (ICG) drugs and an IR light irradiation lead to visualization of cancer cells, IR light is also becoming indispensable as a light source for a medical device from a viewpoint of increasing features.

In a light source for a medical device, a top-hat high-angle distribution, a top-hat illuminance distribution, a high wattage number, and a low etendue are demanded for condensing light to a light guide with respect to visible light and the IR light. From a viewpoint of usability in the medical field, a diameter of the light guide tends to be reduced, and a compatibility between the low etendue and the high wattage is demanded. While an end surface emitting LD is considered as a light source thereof, the LD has a small beam-divergence angle and is asymmetric in an H/V direction. Therefore, it is difficult to obtain the condensed light having the top-hat high angle distribution. Accordingly, there is a problem in that an optical configuration for dealing with an enlargement of the beam-divergence angle of the IR light source and the reduction of an H/V imbalance of the angle distribution becomes complicated.

In contrast, in the present embodiment, the light source unit 11 and the light source unit 12 that respectively output the light having the wavelength of the visible light region (e.g., the blue light Lb) and the wavelength of the near-infrared region (the IR light Lir) to be incident on the light guide 30 are disposed such that the light source unit 12 is disposed before the light source unit 11 to cause an optical path length of IR light Lir to be longer than an optical path length of the blue light Lb and to cause the IR light Lir and the blue light Lb to be incident on the light guide 30 in the coaxial optical path. As a result, the divergence angle of the IR light Lir is increased, the deviation of the angular distribution is reduced, and the illuminance distribution is made uniform.

As described above, according to the present embodiment, it is possible to provide the light source apparatus 1 having the top-hat high angular distribution and the top-hat illuminance distribution.

Further, in the light source apparatus 1 of the present embodiment, as described above, the light source unit 11 that outputs the blue light Lb is disposed between the light source unit 12 that outputs the IR light Lir and the condenser lens 27, for example, making it possible to easily extend the optical path length of IR light Lir. Therefore, it is possible to achieve a reduction in size of the light source apparatus 1.

Further, in the light source apparatus 1 of the present embodiment, the beam shaping element 15 is disposed between the light source unit 12 that outputs the IR light Lir and the condenser lens 27, in particular, at a stage after the lens 122 that constitutes the light source unit 12 and that is close to the light source unit 12. Thus, for example, it is possible to correct the angular distribution of the largely deviated IR light Lir outputted immediately after the light source unit 12 as illustrated in FIG. 4A to the angular distribution in which the deviation is reduced as illustrated in FIG. 4B, at the converging point of the condenser lens 27 disposed before the rod integrator 28. Therefore, it is possible to provide the light source apparatus 1 having a higher top-hat high-angle distribution and a higher top-hat illuminance distribution.

Furthermore, in the light source apparatus 1 of the present embodiment, as described above, the divergence angle of the IR light Lir at the time of reaching the condenser lens 27 is enlarged by the extension of the optical path length of the IR light Lir outputted from the light source unit 12. Therefore, because the IR light Lir is rapidly condensed by the condenser lens 27, the angular distribution of the light applied from the light guide 30 is increased. Therefore, it is possible to provide the light source apparatus 1 having the top-hat high angular distribution.

Further, in the light source apparatus 1 of the present embodiment, because the condenser lens 27 includes a high refractive index material or configured by a combination of a plurality of lenses, the converging point of the IR light Lir is reduced. Therefore, because the amount of light of the IR light Lir entering the rod integrator 28 increases, the light utilization efficiency is improved.

Further, in the light source apparatus 1 of the present embodiment, because the rod integrator 28 is disposed between the condenser lens 27 and the light guide 30, the illuminance distribution of the light applied from the light guide 30 becomes more uniform. Therefore, it is possible to provide the light source apparatus 1 having a more top-hat illuminance distribution.

Furthermore, for example, when the polarization reflection characteristics of an object to be illuminated at an end of the light guide 30 are biased (for example, the S-polarized light has a high reflectance whereas the P-polarized light has a low reflectance, for example), the object to be illuminated appears bright or dark depending on an angle at which the object to be illuminated is observed. In addition, when the degree of polarization is different depending on a color of the illumination light, a white balance of white can change. In contrast, in the light source apparatus 1 of the present embodiment, for example, because the polarizing plate 24 and the quarter wavelength plate 26 are disposed before the condenser lens 27, the polarization of the IR light Lir disturbed by passing through the beam shaping element 15, the reflection mirror 16, the dichroic mirror 18, and the PBS mirror 19 is adjusted and the deviation of the polarization is reduced, making it possible to reduce the brightness at any observation angle. In addition, it is possible to reduce a change in the white balance.

Incidentally, by using the reflection mirror 25 as a PBS mirror, it is possible to eliminate the polarizing plate 24. In addition, it is possible to reduce the quarter wavelength plate 26 with a polarizing scrambler.

Next, modification examples of the present disclosure will be described. Hereinafter, the similar components to those of the embodiment described above are denoted by the same reference numerals, and description thereof is omitted as appropriate.

### 2. Modification Examples

FIG. 6 schematically illustrates a configuration example of a light source apparatus (a light source apparatus 2) according to a modification example of the present disclosure. The light source apparatus 2 is used as a light source of a medical device such as a gastric camera or an endoscope camera, for example, as with the above-described embodiment.

In the above-described embodiment, an example is described in which the light source unit 12 that outputs the blue light Lb as the light source unit that outputs the light having the wavelength in the visible light region, the light source unit 13 that outputs the yellow light Ly, and the light source unit 14 that outputs, for example, the excitation light EL. However, a configuration of the light source unit that outputs the light having the wavelength in the visible light region is not limited thereto.

As illustrated in FIG. 6, the light source apparatus 2 of the present modification example uses a light source unit 41 in which a plurality of solid-state light-emitting elements that respectively outputs red light Lr, green light Lg, and blue light Lb is disposed in an array, so that the red light Lr, the green light Lg, and the blue light Lb are outputted from the light source unit 11 that corresponds to the “first light source unit” of the present disclosure.

The light source apparatus 2 includes, for example, the light source units 11 and 12 and the light guide 30, and further includes the beam shaping elements 15 and 46, the reflection mirrors 16 and 43, the dichroic mirrors 18, 44, and 45, the reduction lens 22, the condenser lens 27, the rod integrator 28, the diffuser plate 29, the light guide 30, the light source unit 41, the half wavelength plate 42, the PBS prism 47, and the quarter wavelength plate 48.

Each member constituting the light source apparatus 2 is disposed as follows. In the light source units 11 and 12, for example, the light source unit 12 and the light source unit 11 are disposed in parallel in this order in the X-axis direction, for example, in a direction (for example, the Y-axis direction) in which the pieces of light (the blue light Lb and the IR light Lir) respectively outputted from the light source units 11 and 12 are respectively reflected by the reflection mirror 16 and the dichroic mirror 18. The beam shaping element 15, the reflection mirror 16, and the dichroic mirror 18 are disposed in this order on the optical path of the IR light Lir outputted from the light source unit 12 as with the above-described embodiment.

In the light source apparatus 2 of the present modification example, for example, the half wavelength plate 42 and the reflection mirror 43 are disposed on the optical path of the red light Lr outputted from the light source unit 41 in the X-axis direction. The red light Lr is reflected by the reflection mirror 43 in the Y-axis direction, enters the PBS prism 47 via the dichroic mirrors 44 and 45 and the reduction lens 22 disposed on the optical path thereof and the beam shaping element 46, and is reflected toward the light source unit 11 at a polarization selection surface of the PBS prism 47. The green light Lg outputted from the light source unit 41 in the X-axis direction is reflected in the Y-axis direction by the dichroic mirror 44, enters the PBS prism 47 via the dichroic mirror 45 and the reduction lens 22 disposed on the optical path thereof and the beam shaping element 46, and is reflected toward the light source unit 11 on the polarization selection surface of the PBS prism 47. The blue light Lb outputted from the light source unit 41 in the X-axis direction is reflected in the Y-axis direction by the dichroic mirror 45, enters PBS prism 47 via the reduction lens 22 and the beam shaping element 46 disposed on the optical path thereof, and is reflected toward the light source unit 11 on the polarization selection surface of the PBS prism 47.

The red light Lr, the green light Lg, and the blue light Lb reflected by the PBS prism 47 are diffused and reflected by the diffusion reflection plate 111 via the quarter wavelength plate 48 and the lens 112. The red light Lr, the green light Lg, and the blue light Lb diffused and reflected by the diffusion reflection plate 111 enter the dichroic mirror 18 via the lens 112, the quarter wavelength plate 48, and the PBS prism 47, are reflected in the Y-axis on the reflection surface 18S, and are multiplexed in an optical path coaxial with the IR light Lir. Thereafter, the multiplexed IR light Lir, the red light Lr, the green light Lg, and the blue light Lb are condensed by the condenser lens 27 and enter the light guide 30 through the rod integrator 28 and the diffuser plate 29.

As described above, regardless of a configuration of an optical system of the light source unit 11 that outputs the light having the wavelength in the visible light region, the light source unit 11 that outputs the light having the wavelength in the visible light region is disposed between the light source unit 12 that outputs the IR light Lir and, for example, the condenser lens 27, making it possible to obtain similar effects to those of the above-described embodiment.

Although the present technology has been described with reference to the embodiments and the modification examples, the present technology is not limited to the above-described embodiments and the like, and various modifications are possible. For example, the arrangement and the number of the components of the optical system exemplified in the above-described embodiments and the like are merely examples, and it is not necessary to include all the components, and other components may be further included.

Further, the light source apparatus 1 or 2 of the present technology may be used in an electronic device other than the medical device. For example, the light source apparatus 1 or 2 may be used as illumination applications, and is applicable to, for example, headlamps of automobiles and light sources for light-up.

The effects described herein are not necessarily limited, and may be any of the effects described in the present disclosure.

It should be noted that the present technology may have the following configuration. According to the present technology of the following configuration, a first light source unit and a second light source unit that respectively output first light having a wavelength in a visible light region and second light having a wavelength in a near-infrared region to be incident on a light guide are disposed before the first light source to cause an optical path length of the second light to be longer than an optical path length of the first light and to cause the first light and the second light to be incident on the light guide coaxially. Accordingly, the divergence angle of the second light is enlarged, and it is possible to provide a light source apparatus having a top-hat high angle distribution and a top-hat illuminance distribution.

(1)

A light source apparatus including:
a first light source unit that outputs first light having a wavelength in a visible light region;
a second light source unit that is disposed before the first light source unit, and outputs second light having a wavelength in a near-infrared region; and
a light guide on which the first light and the second light are incident, in which
the first light and the second light are incident on the light guide in a coaxial optical path.
   (2)

The light source apparatus according to (1), further including a beam shaping element that is disposed between the second light source unit and the first light source unit, and controls an angular distribution of the second light.

(3)

The light source apparatus according to (1) or (2), further including one or more condenser lenses disposed between the first light source unit and the light guide.

(4)

The light source apparatus according to (3), further including an integrator optical system disposed between the light guide and the one or more condenser lenses.

(5)

The light source apparatus according to (3) or (4), further including a polarizing optical element disposed between the first light source unit and the one or more condenser lenses.

(6)

The light source apparatus according to any one of (2) to (5), further including:
a reflection mirror that is disposed after the beam shaping element and reflects the second light in a first direction; and
a first synthesis mirror that is disposed on an optical path of the second light and multiplexes the first light and the second light by transmitting the second light and selectively reflecting the first light in the first direction.
   (7)

The light source apparatus according to (6), in which the first synthesis mirror is a dichroic mirror.

(8)

The light source apparatus according to any one of (1) to (7), in which the first light source unit and the second light source unit are linearly disposed.

(9)

The light source apparatus according to any one of (1) to (8), in which
the second light source unit includes one or more light emitting elements, and
the one or more light emitting elements are semiconductor lasers.
   (10)

The light source apparatus according to (3) to (9), in which the one or more condenser lenses include a high refractive index material.

(11)

The light source apparatus according to any one of (6) to (10), in which at least one of a light amount sensor or a chromaticity sensor is disposed on a surface, of the reflection mirror, on an opposite side of a reflection surface of the reflection mirror.

(12)

The light source apparatus according to any one of (3) to 11), further including a third light source unit that is disposed between the first light source unit and the one or more condenser lenses, and outputs third light having a wavelength in a visible light region.

(13)

The light source apparatus according to (12), further including a second synthesis mirror that is disposed on an optical path of the first light and the second light that are multiplexed, and multiplexes the first light, the second light, and the third light by transmitting the third light and selectively reflecting the first light and the second light in a second direction.

(14)

The light source apparatus according to (13), in which the second synthesis mirror is a polarizing beam splitter.

(15)

The light source apparatus according to any one of (12) to (14), in which the first light is blue light and the third light is yellow light.

(16)

The light source apparatus according to any one of (12) to (15), further including a fourth light source unit that outputs excitation light, in which
the third light source unit includes a wavelength conversion unit that outputs the third light by performing a wavelength conversion on the excitation light, and
the first light source unit, the second light source unit, the third light source unit, and the fourth light source unit are linearly disposed.

The present application claims the benefit of Japanese Priority Patent Application JP2022-096759 filed with the Japan Patent Office on Jun. 15, 2022, the entire contents of which are incorporated herein by reference.

It should be understood by those skilled in the art that various modifications, combinations, sub-combinations and alterations may occur depending on design requirements and other factors insofar as they are within the scope of the appended claims or the equivalents thereof.

What is claimed is:

1. A light source apparatus, comprising:
a first light source unit that outputs first light having a wavelength in a visible light region;
a second light source unit that is disposed before the first light source unit, and outputs second light having a wavelength in a near-infrared region; and
a light guide on which the first light and the second light are incident, wherein
the first light and the second light are incident on the light guide in a coaxial optical path.

2. The light source apparatus according to claim 1, further comprising a beam shaping element that is disposed between the second light source unit and the first light source unit, and controls an angular distribution of the second light.

3. The light source apparatus according to claim 1, further comprising one or more condenser lenses disposed between the first light source unit and the light guide.

4. The light source apparatus according to claim 3, further comprising an integrator optical system disposed between the light guide and the one or more condenser lenses.

5. The light source apparatus according to claim 3, further comprising a polarizing optical element disposed between the first light source unit and the one or more condenser lenses.

6. The light source apparatus according to claim 2, further comprising:
a reflection mirror that is disposed after the beam shaping element and reflects the second light in a first direction; and
a first synthesis mirror that is disposed on an optical path of the second light and multiplexes the first light and the second light by transmitting the second light and selectively reflecting the first light in the first direction.

7. The light source apparatus according to claim 6, wherein the first synthesis mirror comprises a dichroic mirror.

8. The light source apparatus according to claim 1, wherein the first light source unit and the second light source unit are linearly disposed.

9. The light source apparatus according to claim 1, wherein
the second light source unit includes one or more light emitting elements, and
the one or more light emitting elements comprise semiconductor lasers.

10. The light source apparatus according to claim 3, wherein the one or more condenser lenses include a high refractive index material.

11. The light source apparatus according to claim 6, wherein at least one of a light amount sensor or a chromaticity sensor is disposed on a surface, of the reflection mirror, on an opposite side of a reflection surface of the reflection mirror.

12. The light source apparatus according to claim 3, further comprising a third light source unit that is disposed between the first light source unit and the one or more condenser lenses, and outputs third light having a wavelength in a visible light region.

13. The light source apparatus according to claim 12, further comprising a second synthesis mirror that is disposed on an optical path of the first light and the second light that are multiplexed, and multiplexes the first light, the second light, and the third light by transmitting the third light and selectively reflecting the first light and the second light in a second direction.

14. The light source apparatus according to claim 13, wherein the second synthesis mirror comprises a polarizing beam splitter.

15. The light source apparatus according to claim 12, wherein the first light comprises blue light and the third light comprises yellow light.

16. The light source apparatus according to claim 12, further comprising a fourth light source unit that outputs excitation light, wherein
the third light source unit includes a wavelength conversion unit that outputs the third light by performing a wavelength conversion on the excitation light, and
the first light source unit, the second light source unit, the third light source unit, and the fourth light source unit are linearly disposed.
